(19) United States
(12) Patent Application Publication
(10) Pub. No.: US 2024/0274287 A1
(43) Pub. Date: Aug. 15, 2024

(54) IMAGE DIAGNOSIS ASSISTING DEVICE, OPERATION METHOD OF IMAGE DIAGNOSIS ASSISTING DEVICE, AND A PROGRAM

(71) Applicant: FUJIFILM Corporation, Tokyo (JP)

(72) Inventor: Aya OGASAWARA, Tokyo (JP)

(73) Assignee: FUJIFILM Corporation, Tokyo (JP)

(21) Appl. No.: 18/433,422

(22) Filed: Feb. 6, 2024

(30) Foreign Application Priority Data
Feb. 14, 2023 (JP) 2023-020967

(51) Int. Cl. G16H 50/20; A61B 5/00; G06V 20/70; G16H 10/60; G16H 15/00; G16H 40/67

(52) U.S. Cl. CPC G16H 50/20; A61B 5/4848; A61B 5/742; G06V 20/70; G16H 10/60; G16H 15/00; G16H 40/67; G06V 2201/03

(57) ABSTRACT

Provided are an image diagnosis assisting device, an operation method of an image diagnosis assisting device, and a program that can ascertain a treatment history using information regarding a past treatment location obtained from a medical image, an interpretation report, and the like. An image diagnosis assisting device acquires a first medical image generated by imaging a subject in a latest examination, acquires treatment information regarding a treatment of the subject generates treatment progress information regarding a past treatment location in the subject based on the treatment information, and performs control to display the treatment progress information on a screen on which the first medical image is displayed.

### CROSS-REFERENCE TO RELATED APPLICATION

The present application claims priority under 35 U.S.C § 119(a) to Japanese Patent Application No. 2023-020967 filed on Feb. 14, 2023, which is hereby expressly incorporated by reference, in its entirety, into the present application.

### BACKGROUND OF THE INVENTION

### 1. Field of the Invention

The present invention relates to an image diagnosis assisting device, an operation method of an image diagnosis assisting device, and a program.

### 2. Description of the Related Art

In creating an interpretation report (radiology report) of a medical image for a patient who has a treatment history, the history or the like of a treatment location may be described. The same applies even in a case where an anatomical structure of a current examination target is different from an anatomical structure having a treatment history.

WO2020/188682A discloses a diagnosis support device that uses at least one of physical information including one or more pieces of information capable of estimating a state of an organ to be diagnosed of an examination target subject, which is a subject, or an endoscopic image obtained by imaging the organ to be diagnosed to extract a lesion in the organ to be diagnosed.

JP2009-230609A discloses a medical image diagnosis support device that refers to a dental diagnosis history of a treatment target. The device disclosed in JP2009-230609A displays image information for dentists and tooth position information on a display device, creates a diagnostic history for display related to a part designated by an operator, and displays the diagnostic history for display.

JP2009-125133A discloses a dental care support system that exchanges dental treatment details between a dental clinic and an advanced medical hospital. In the system disclosed in JP2009-125133A, a terminal at a dental clinic and a terminal at an advanced medical hospital are communicatively connected via a network, and in response to a request for advice from the dental clinic to the advanced medical hospital, medical advice information is transmitted from the terminal at the advanced medical hospital to the terminal at the dental clinic.

### SUMMARY OF THE INVENTION

However, the description of the history of the treatment location and the like in the interpretation report places a burden on a radiologist. The reason is as follows. In a case where the treatment progresses successfully, a treatment location such as a lesion will be reduced, disappear, or be excised as a result of the progress of the treatment. In this case, it becomes difficult to specify the treatment location due to, for example, a reduction of the treatment location in the medical image during the treatment and after the treatment. In a case where it is difficult to specify the treatment location in the current image, it is necessary to refer to past medical images, past interpretation reports, and the like.

In addition, in a case where an anatomical structure of an organ or the like for which an instruction of any treatment has been provided at a past point in time is different from an anatomical structure for the purpose of the current examination, in the current examination, it is necessary to refer to a medical image that shows the anatomical structure for which an instruction of a treatment has been provided in the past, to interpret the medical image, and to generate a current interpretation report.

Although the device disclosed in WO2020/188682A, the device disclosed in JP2009-230609A, and the system disclosed in JP2009-125133A all use past data such as a treatment history to support diagnosis, but are not intended to assist in image interpretation.

The present invention has been made in view of such circumstances, and an object of the present invention is to provide an image diagnosis assisting device, an operation method of an image diagnosis assisting device, and a program that can ascertain a treatment history using information regarding a past treatment location obtained from a medical image, an interpretation report, and the like.

An image diagnosis assisting device according to a first aspect of the present disclosure is an image diagnosis assisting device comprising: one or more processors; and one or more memories in which instructions to be executed by the one or more processors are stored, in which the one or more processors are configured to: acquire a first medical image generated by imaging a subject in a latest examination; acquire treatment information regarding a treatment of the subject; generate treatment progress information regarding a past treatment location in the subject based on the treatment information; and perform control to display the treatment progress information on a screen on which the first medical image is displayed.

With the image diagnosis assisting device according to the first aspect of the present disclosure, the treatment progress information regarding the past treatment location is generated and displayed for the first medical image generated in the latest examination. This makes it possible to ascertain the progress of treatment using information regarding the past treatment location.

The latest examination can be understood as a current examination. The current examination can be understood as a present examination.

The first medical image may be a two-dimensional image or a three-dimensional image. The first medical image may be a reconstructed image group including a plurality of reconstructed images.

The one or more processors may acquire a second medical image generated in the past examination for the latest examination.

With an image diagnosis assisting device according to a second aspect, in the image diagnosis assisting device according to the first aspect, the one or more processors may be configured to acquire information regarding the past treatment location of the subject as the treatment information.

According to this aspect, treatment progress information can be generated based on past information regarding the past treatment location of the subject.

With an image diagnosis assisting device according to a third aspect, in the image diagnosis assisting device according to the second aspect, the one or more processors may be configured to acquire, as the information regarding the past treatment location of the subject, character information including a description regarding the past treatment location of the subject.

According to this aspect, treatment progress information can be generated based on character information regarding the past treatment location of the subject.

With an image diagnosis assisting device according to a fourth aspect, in the image diagnosis assisting device according to any one of the first to third aspects, the one or more processors may be configured to acquire request information for the latest examination of the subject as the treatment information.

According to this aspect, treatment progress information can be generated based on current information such as request information for the latest examination of the subject.

With an image diagnosis assisting device according to a fifth aspect, in the image diagnosis assisting device according to the fourth aspect, the one or more processors may be configured to acquire, as the request information for the latest examination of the subject, character information including a description regarding a purpose of the latest examination for the subject.

According to this aspect, treatment progress information can be generated based on character information including a description regarding the purpose of the latest examination.

With an image diagnosis assisting device according to a sixth aspect, in the image diagnosis assisting device according to any one of the first to fifth aspects, the one or more processors may be configured to acquire, as the treatment information, at least one of a name of an anatomical structure for which an instruction of a treatment has been provided in the past, a name of a lesion for which the instruction of the treatment has been provided in the past, a position of the lesion for which the instruction of the treatment has been provided in the past, or a treatment method that has been performed in the past.

According to this aspect, treatment progress information can be generated based on the name of an anatomical structure for which an instruction of a treatment has been provided in the past, and the like.

With an image diagnosis assisting device according to a seventh aspect, in the image diagnosis assisting device according to any one of the first to sixth aspects, the one or more processors may be configured to display the treatment progress information on a first diagnosis target image included in the first medical image, the first diagnosis target image including a first anatomical structure to be diagnosed in the latest examination.

According to this aspect, it is possible to ascertain the past treatment history represented by the treatment progress information in the first diagnosis target image.

With an image diagnosis assisting device according to an eighth aspect, in the image diagnosis assisting device according to the seventh aspect, the one or more processors may be configured to generate, as the treatment progress information, information for designating, in a reconstructed image group including a plurality of two-dimensional reconstructed images, a position of the reconstructed image including the past treatment location.

According to this aspect, the position of past treatment can be specified in the first diagnosis target image.

With an image diagnosis assisting device according to a ninth aspect, in the image diagnosis assisting device according to the seventh aspect, the one or more processors may be configured to apply segmentation processing of the first anatomical structure or labeling processing of the first anatomical structure to the first medical image to specify the past treatment location in the first diagnosis target image.

In this aspect, a trained learning model that implements segmentation processing or a trained learning model that implements labeling processing may be applied.

With an image diagnosis assisting device according to a tenth aspect, in the image diagnosis assisting device according to any one of the seventh to ninth aspects, the one or more processors may be configured to: acquire a second medical image generated by imaging a subject in a past examination; and apply segmentation processing of the first anatomical structure or labeling processing of the first anatomical structure to the second medical image to specify the past treatment location in the second medical image.

According to this aspect, a past treatment location can be specified from the second medical image generated in the past examination.

With an image diagnosis assisting device according to an eleventh aspect, in the image diagnosis assisting device according to any one of the first to sixth aspects, the one or more processors may be configured to generate, as the treatment progress information, a first trimmed image in which a region corresponding to the past treatment location in a first diagnosis target image included in the first medical image is trimmed, the first diagnosis target image including a first anatomical structure to be diagnosed in the latest examination.

According to this aspect, it is possible to ascertain the feature of the past treatment location in the first medical image.

With an image diagnosis assisting device according to a twelfth aspect, in the image diagnosis assisting device according to the eleventh aspect, the one or more processors may be configured to: analyze the first trimmed image; in a case where a feature in an image related to the treatment location is not recognizable in the first trimmed image, generate a second trimmed image in which the region corresponding to the treatment location is trimmed from a past medical image of the subject; and display the second trimmed image in the region corresponding to the past treatment location in the first diagnosis target image.

According to this aspect, it is possible to ascertain the feature of the past treatment location in the first medical image.

With an image diagnosis assisting device according to a thirteenth aspect, in the image diagnosis assisting device according to any one of the first to twelfth aspects, the one or more processors may be configured to: in a case where a first anatomical structure to be examined in the latest examination is different from a second anatomical structure which is the past treatment location of the subject, acquire the treatment information for the second anatomical structure; generate the treatment progress information for the second anatomical structure based on the treatment information for the second anatomical structure; and display the treatment progress information for the second anatomical structure on a second diagnosis target image included in the first medical image, the second diagnosis target image including the second anatomical structure.

According to this aspect, even in a case where the anatomical structure of the examination target in the latest examination is different from the anatomical structure of the examination target in the past examination, the past treatment history can be ascertained in the second diagnosis target image.

With an image diagnosis assisting device according to a fourteenth aspect, in the image diagnosis assisting device according to any one of the first to thirteenth aspects, the one or more processors may be configured to: generate a plurality of pieces of the treatment progress information; and apply a different display mode to each of the plurality of pieces of treatment progress information.

According to this aspect, even in a case where there are a plurality of treatment histories, each of the plurality of treatment histories can be distinguished.

An operation method of an image diagnosis assisting device according to a fifteenth aspect of the present disclosure is an operation method of an image diagnosis assisting device to which a computer is applied, the operation method comprising: by the image diagnosis assisting device, acquiring a first medical image generated by imaging a subject in a latest examination; acquiring treatment information regarding a treatment of the subject; generating treatment progress information regarding a past treatment location in the subject based on the treatment information; and performing control to display the treatment progress information on a screen on which the first medical image is displayed.

With the operation method of the image diagnosis assisting device according to the fifteenth aspect of the present disclosure, it is possible to obtain the same effects as the image diagnosis assisting device according to the first aspect of the present disclosure.

In the operation method of the image diagnosis assisting device according to the fifteenth aspect, the same items as those specified in the second to fourteenth aspects can be combined as appropriate. In that case, the component responsible for the specified processing or function in the image diagnosis assisting device can be understood as a component of the operation method of the image diagnosis assisting device responsible for the corresponding processing or function.

A program according to a sixteenth aspect of the present disclosure is a program causing a computer to implement: a function of acquiring a first medical image generated by imaging a subject in a latest examination; a function of acquiring treatment information regarding a treatment of the subject; a function of generating treatment progress information regarding a past treatment location in the subject based on the treatment information; and a function of performing control to display the treatment progress information on a screen on which the first medical image is displayed.

With the program according to the sixteenth aspect of the present disclosure, it is possible to obtain the same effects as the image diagnosis assisting device according to the first aspect of the present disclosure.

In the program according to the sixteenth aspect, the same items as those specified in the second to fourteenth aspects can be combined as appropriate. In that case, the component responsible for the specified processing or function in the image diagnosis assisting device can be understood as a component of the program responsible for the corresponding processing or function.

According to the aspects of the present invention, the treatment progress information regarding the past treatment location is generated and displayed for the first medical image generated in the latest examination. This makes it possible to ascertain the progress of treatment using information regarding the past treatment location.

### BRIEF DESCRIPTION OF THE DRAWINGS

FIG. 1 is an overall configuration diagram of a hospital system.

FIG. 2 is a functional block diagram showing an electric configuration of a server device shown in FIG. 1.

FIG. 3 is a block diagram showing an example of a hardware configuration of the server device shown in FIG. 2.

FIG. 4 is a flowchart showing a procedure of an image diagnosis assisting method according to an embodiment.

FIG. 5 is a schematic diagram of treatment progress information according to a first specific example.

FIG. 6 is a schematic diagram of treatment progress information according to a second specific example.

FIG. 7 is a schematic diagram of treatment progress information according to a third specific example.

FIG. 8 is a schematic diagram of acquisition of treatment information according to the first specific example.

FIG. 9 is a schematic diagram of acquisition of treatment information according to the second specific example.

FIG. 10 is a schematic diagram of acquisition of treatment information according to the third specific example.

FIG. 11 is a schematic diagram of an image diagnosis assisting method according to a modification example.

### DESCRIPTION OF THE PREFERRED EMBODIMENTS

Hereinafter, a preferred embodiment of the present invention will be described in detail with reference to the accompanying drawings. In the present specification, the same components are denoted by the same reference numerals, and duplicate description thereof will be omitted as appropriate.

### Configuration Example of Hospital System

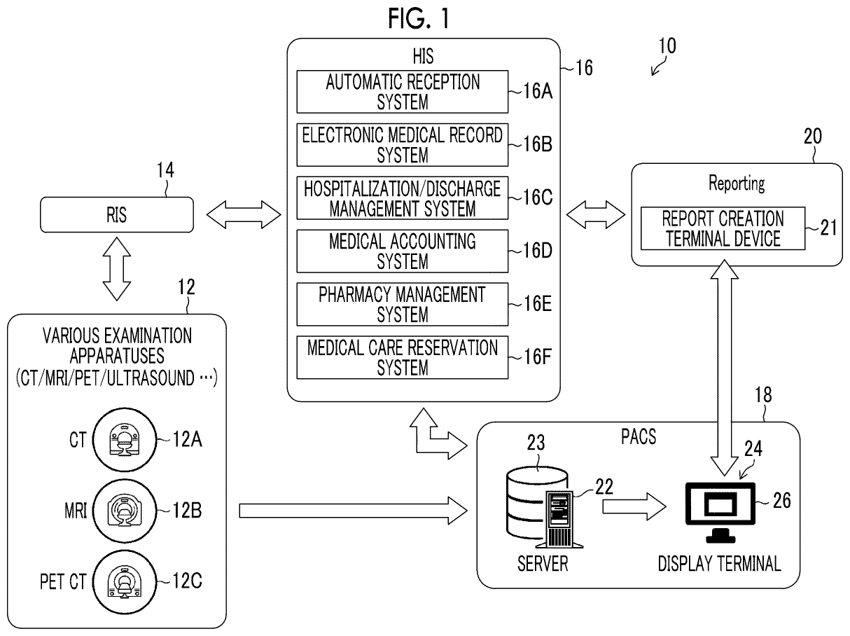

FIG. 1 is an overall configuration diagram of a hospital system. Various systems constituting a hospital system 10 shown in FIG. 1 include one or more computers, and the one or more computers execute various programs stored on tangible computer-readable mediums to implement one or more functions in the various systems.

The hospital system 10 comprises various examination apparatuses 12. The examination apparatus 12 images an examination target subject, which is a subject, and generates a medical image. In FIG. 1, a CT apparatus 12A, an MRI apparatus 12B, and a PETCT apparatus 12C are exemplified as the examination apparatus 12. Other examples of the examination apparatus 12 include an X-ray imaging apparatus, an ultrasound apparatus, a CR apparatus using a flat X-ray detector, and the like.

Note that, CT is an abbreviation for Computed Tomography. MRI is an abbreviation for Magnetic Resonance Imaging. PET is an abbreviation for Positron Emission Tomography. CR is an abbreviation for Computed Radiography. The examination apparatus 12 may also be referred to as a modality.

The hospital system 10 comprises a radiology information system 14. The radiology information system 14 mainly performs management from reservation of examinations and treatments in which radiation equipment is used to examination results. The radiology information system 14 stores medical images generated in examinations, past interpretation reports, interpretation reports created in a current examination, and the like. The radiology information system 14 may be represented using RIS, which is an abbreviation for Radiology Information Systems.

The hospital system 10 comprises a hospital information system 16. The hospital information system 16 is a general term for various information systems in a hospital. In general, the hospital information system 16 may include an automatic reception system 16A, an electronic medical record system 16B, a hospitalization/discharge management system 16C, a medical accounting system 16D, a pharmacy management system 16E, a medical care reservation system 16F, and the like.

The hospital information system 16 stores all information regarding the hospital. For example, the electronic medical record system 16B stores a surgical history, a treatment history, and the like of each patient as an electronic medical record. The hospital information system 16 may be represented using an abbreviation HIS for Hospital Information Systems.

The hospital system 10 comprises an image storage communication system 18. The image storage communication system 18 comprises a server device 22 and a display terminal 24. The server device 22 is provided with a database 23. The display terminal 24 is provided with a display device 26.

The image storage communication system 18 receives a medical image transmitted from the examination apparatus 12. The received medical image is stored in the database 23. The image storage communication system 18 reads out the medical image stored in the database 23. The read-out medical image is displayed on the display device 26 of the display terminal 24. DICOM is applied as a standard for storage and communication of medical images managed using the server device 22, the database 23, and the display terminal 24. DICOM is an abbreviation for Digital Imaging and COmmunications in Medicine. The server device 22 may be referred to as a DICOM server.

A radiologist can visually recognize the medical image to be diagnosed using the display terminal 24. In addition, the radiologist can check the content of the orders transmitted from various medical departments using the display terminal 24. Furthermore, the radiologist can ascertain the purpose of the examination based on the orders transmitted from various medical departments. In addition, the image storage communication system 18 may be represented using PACS, which is an abbreviation for Picture Archiving and Communication Systems.

The hospital system 10 comprises a report creation system 20. The report creation system 20 comprises a report creation terminal device 21 that is operated by a radiologist to create an interpretation report. The report creation terminal device 21 may store a program for supporting creation of the interpretation report, and execute the program for supporting the creation of the interpretation report.

The examination apparatus 12 and the like constituting the hospital system 10 are electrically connected to each other via an in-hospital network in a communicable manner. An in-hospital network LAN can be applied. The in-hospital network may be wired or wireless.

The in-hospital network may be connected to a public line network such as the Internet via a router (not shown). That is, the hospital system 10 may be electrically connected to an external device, such as a cloud server, via an in-hospital network and a public line network in a communicable manner. LAN is an abbreviation for Local Area Network.

### Electric Configuration of Server Device

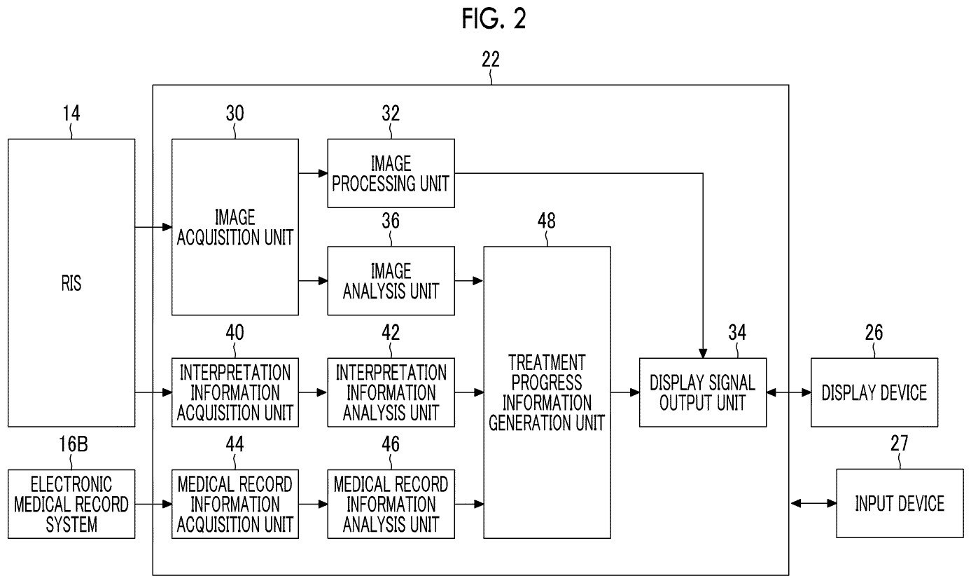

FIG. 2 is a functional block diagram showing the server device shown in FIG. 1. The server device 22 shown in FIG. 2 functions as an image diagnosis assisting device. The server device 22 comprises an image acquisition unit 30, an image processing unit 32, and a display signal output unit 34.

The image acquisition unit 30 acquires a current image that is a medical image stored in the radiology information system 14 and that is a medical image to be diagnosed in a current examination target subject. The image acquisition unit 30 also acquires a past image which is a medical image in a past examination of the current examination target subject.

Here, the current examination is understood as the latest examination in a plurality of examinations in time series. The past examination is understood as one or more past examinations excluding the latest examination in a plurality of examinations in time series. The current medical image is understood as the latest medical image.

In addition, the current image described in the embodiment is an example of a first medical image. The past image described in the embodiment is an example of a second medical image.

The image processing unit 32 performs various types of image processing on the medical image acquired using the image acquisition unit 30. Examples of various types of image processing include processing corresponding to a display condition of the display device 26, such as resolution conversion processing and size conversion processing. The display signal output unit 34 transmits a display signal representing the medical image subjected to image processing using the image processing unit 32 to the display device 26 of the display terminal 24.

The server device 22 comprises an image analysis unit 36. The image analysis unit 36 analyzes the medical image acquired using the image acquisition unit 30. Examples of the analysis of a medical image include segmentation processing and labeling processing of an anatomical structure such as an organ. Further, other examples of the analysis of a medical image include segmentation processing and labeling processing of an abnormal region for each anatomical structure. Examples of an anatomical structure include organs, muscles, tendons, joints, bones, nerves, and blood vessels.

A trained learning model may be applied to the image analysis unit 36. The learning model applied to the image analysis unit 36 is a recognition model that recognizes one or more anatomical structures or an abnormal region for each anatomical structure from one medical image, and a learning model that outputs a recognition result of an anatomical structure or the like in a case where a medical image is input, using a set of the medical image and the recognition result of the anatomical structure or the like as learning data, may be applied. Deep learning may be applied to the training of the learning model applied to the image analysis unit 36.

Further, a learning model that performs labeling of one or more anatomical structures or the like from one medical image may be applied to the image analysis unit 36. That is, a learning model that outputs a label such as an anatomical structure in a case where the medical image is input, using a set of the medical image and the label such as the anatomical structure as learning data, may be applied.

The server device 22 comprises an interpretation information acquisition unit 40 and an interpretation information analysis unit 42. The interpretation information acquisition unit 40 acquires a past interpretation report for the current examination target subject from among the past interpretation reports stored in the radiology information system 14.

The interpretation information analysis unit 42 analyzes the interpretation report acquired using the interpretation information acquisition unit 40 to acquire first treatment information. For example, the interpretation information analysis unit 42 analyzes sentences described in the acquired interpretation report to extract first treatment information such as a creation date and time of the interpretation report, names of organs, names of lesions, positions of the lesions, and treatment methods. The interpretation information acquisition unit 40 and the interpretation information analysis unit 42 described in the embodiment are examples of components of a treatment information acquisition unit.

The server device 22 comprises a medical record information acquisition unit 44 and a medical record information analysis unit 46. The medical record information acquisition unit 44 acquires the electronic medical record for the current examination target subject from among the electronic medical records stored in the electronic medical record system 16B provided in the hospital information system 16 shown in FIG. 1.

The medical record information analysis unit 46 analyzes sentences described in the electronic medical record acquired using the medical record information acquisition unit 44 to extract second treatment information such as a date and time of the past treatment and treatment details. The medical record information acquisition unit 44 and the medical record information analysis unit 46 described in the embodiment are examples of components of the treatment information acquisition unit.

The server device 22 comprises a treatment progress information generation unit 48. The treatment progress information generation unit 48 acquires the first treatment information from the interpretation information analysis unit 42. Further, the treatment progress information generation unit 48 acquires the second treatment information from the medical record information analysis unit 46. The treatment progress information generation unit 48 generates treatment progress information indicating an abnormal region that has been extracted in a past image based on treatment information including first treatment information and second treatment information and that has disappeared or has been reduced in the current image. The treatment progress information generation unit 48 transmits the treatment progress information to the display signal output unit 34.

The treatment progress information generation unit 48 may acquire at least one of the first treatment information or the second treatment information in a case of generating the treatment progress information, and may generate the treatment progress information using the acquired treatment information.

The display signal output unit 34 generates a display signal representing treatment progress information and transmits the display signal representing treatment progress information to the display device 26. The display device 26 displays the treatment progress information on a screen on which the current image is displayed.

The server device 22 acquires input information transmitted from an input device 27 provided in the display terminal 24 shown in FIG. 1. A keyboard, a mouse, or the like is applied to the input device 27. An operator of the display terminal 24 can operate the input device 27 to transmit desired information to the server device 22.

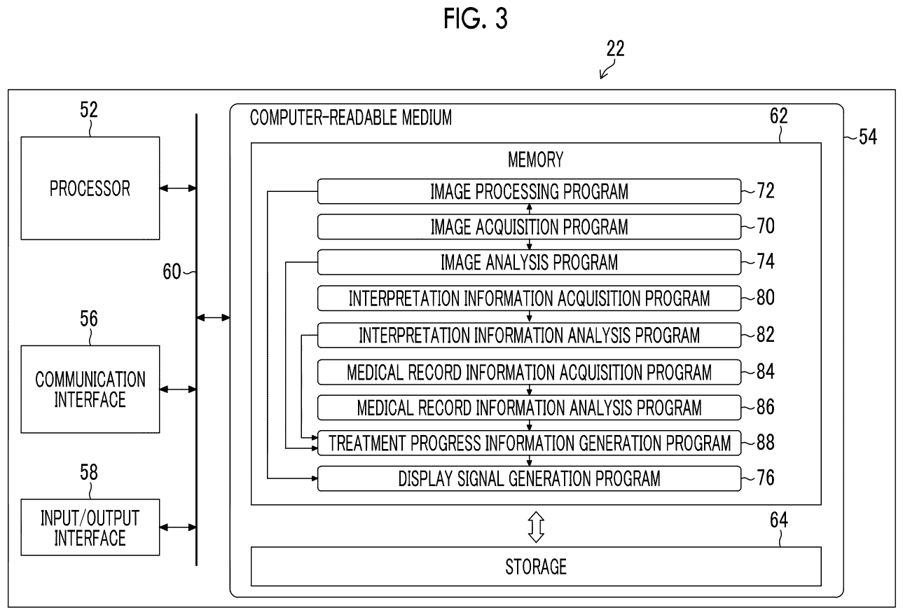

FIG. 3 is a block diagram schematically showing an example of a hardware configuration of the server device shown in FIG. 2. The server device 22 comprises one or more processors 52 and one or more memories 62. The server device 22 comprises a communication interface 56 and an input/output interface 58.

The processor 52 executes various programs stored in the memory 62 of a computer-readable medium 54 to implement various functions of the server device 22. The processor 52 includes a central processing unit (CPU). The processor 52 may include a graphics processing unit (GPU). The processor 52 is connected to the computer-readable medium 54, the communication interface 56, and the input/output interface 58 via a bus 60.

The computer-readable medium 54 includes a memory 62 which is a main memory and a storage 64 which is an auxiliary memory. A semiconductor memory, a hard disk apparatus, a solid-state drive apparatus, and the like may be applied to the computer-readable medium 54. Any combination of a plurality of devices may be applied to the computer-readable medium 54.

A hard disk apparatus may be referred to as an HDD, which is an abbreviation for Hard Disk Drive in English. A solid-state drive apparatus may be referred to as an SSD, which is an abbreviation for Solid-State Drive in English.

The memory 62 of the computer-readable medium 54 stores an image acquisition program 70, an image processing program 72, an image analysis program 74, and a display signal output program 76. The memory 62 also stores an interpretation information acquisition program 80, an interpretation information analysis program 82, a medical record information acquisition program 84, a medical record information analysis program 86, and a treatment progress information generation program 88.

The image acquisition program 70 is applied to the image acquisition unit 30 shown in FIG. 2 to implement an image acquisition function. The image processing program 72 is applied to the image processing unit 32 to implement an image processing function using a processing result of the image acquisition program 70. The image analysis program 74 is applied to the image analysis unit 36 to implement an image analysis function using the processing result of the image acquisition program 70.

The interpretation information acquisition program 80 is applied to the interpretation information acquisition unit 40 to implement an interpretation information acquisition function. The interpretation information analysis program 82 is applied to the interpretation information analysis unit 42 to implement an interpretation information analysis function using a processing result of the interpretation information acquisition program 80. The interpretation information analysis function is a first treatment information acquisition function, and is understood as a component of a treatment information acquisition function.

The medical record information acquisition program 84 is applied to the medical record information acquisition unit 44 to implement a medical record information acquisition function. The medical record information analysis program 86 is applied to the medical record information analysis unit 46 to implement a medical record information analysis function using a processing result of the medical record information analysis program 86. The medical record information analysis function is a second treatment information acquisition function, and is understood as a component of the treatment information acquisition function.

The treatment progress information generation program 88 is applied to the treatment progress information generation unit 48. The treatment progress information generation program 88 implements a treatment progress information generation function using the first treatment information that is a processing result of the image analysis program 74 and a processing result of the interpretation information analysis program 82 and the second treatment information that is the processing result of the medical record information analysis program 86.

The display signal output program 76 is applied to the display signal output unit 34. The display signal output program 76 implements a display signal output function using a processing result of the image processing program 72. The display signal output program 76 also implements a display signal output function in a case of displaying the treatment progress information using a processing result of the treatment progress information generation program 88.

Various programs stored in the computer-readable medium 54 include one or more instructions. The computer-readable medium 54 stores various types of data, various parameters, and the like. Note that the term “program” is synonymous with the term “software”.

The hardware structure of the processor 52 is various processors as shown below. The various processors include a central processing unit (CPU) that is a general-purpose processor that acts as various functional units by executing software (programs), a programmable logic device (PLD) that is a processor whose circuit configuration can be changed after manufacture, such as a graphics processing unit (GPU) and a field-programmable gate array (FPGA) which are processors specialized for image processing, a dedicated electrical circuit that is a processor having a circuit configuration designed exclusively for executing specific processing such as an application-specific integrated circuit (ASIC), and the like.

One processing unit may be configured by one of the various processors, or may be configured by the same or different types of two or more processors (for example, a plurality of FPGAs, a combination of a CPU and an FPGA, or a combination of a CPU and a GPU). In addition, a plurality of functional units may be configured by one processor. As an example of configuring a plurality of functional units via one processor, first, as represented by a computer, such as a client or a server, there is a form in which one processor is configured by a combination of one or more CPUs and software, and this processor acts as a plurality of functional units. Second, as represented by a system-on-chip (SoC) or the like, there is a form of using a processor for implementing the function of the entire system including a plurality of functional units with one integrated circuit (IC) chip. Thus, various functional units are configured by using one or more of the above-described various processors as hardware structures.

Furthermore, the hardware structure of these various processors is, more specifically, an electrical circuit (circuitry) in which circuit elements such as semiconductor elements are combined.

The memory 62 stores instructions for execution by the processor 52. The memory 62 includes a random-access memory (RAM) and a read-only memory (ROM) (which are not shown). The processor 52 uses the RAM as a work area, executes software using parameters and various programs including an image processing program including the image acquisition program 70 and the like stored in the ROM, and uses the parameters stored in the ROM or the like to execute various types of processing of the server device 22.

The display terminal 24 shown in FIG. 1 may implement an information processing function in the server device 22. That is, the display terminal 24 may comprise various processing units such as the image acquisition unit 30 shown in FIG. 3 to implement functions of various processing units. In addition, the hospital system 10 shown in FIG. 1 may be provided with an information processing apparatus comprising various processing units such as the image acquisition unit 30 shown in FIG. 3, in addition to the server device 22 and the display terminal 24.

### Procedure of Image Diagnosis Assisting Method According to Embodiment

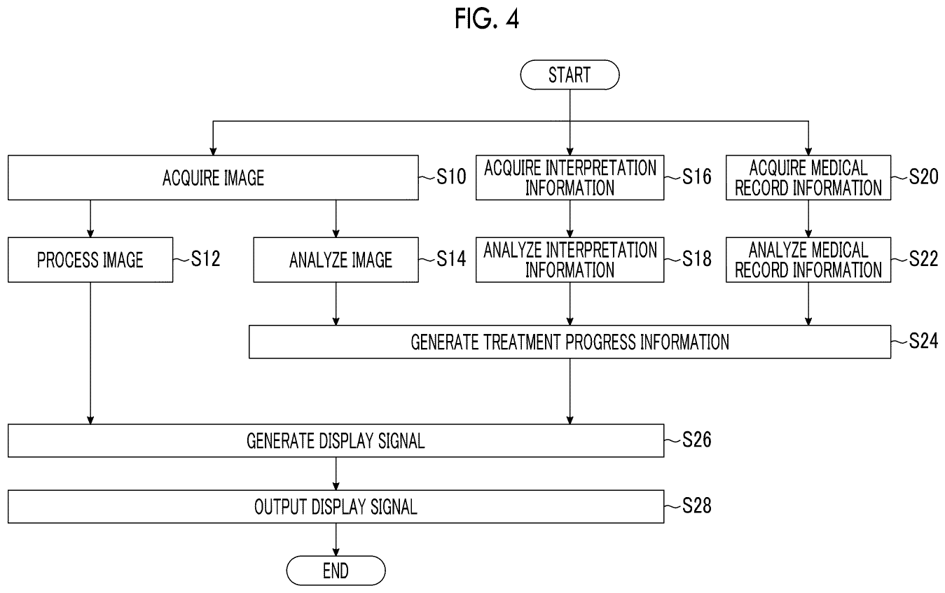

FIG. 4 is a flowchart showing a procedure of an image diagnosis assisting method according to the embodiment. Each step of the image diagnosis assisting method shown in FIG. 4 is executed using various processing units provided in the server device 22 to which a computer is applied. The image diagnosis assisting method is an example of an operation method of an image diagnosis assisting device.

In an image acquisition step S10, the image acquisition unit 30 shown in FIG. 2 acquires a current image and a past image. After the image acquisition step S10, the process proceeds to an image processing step S12 and an image analysis step S14. In the image processing step S12, the image processing unit 32 performs various types of image processing on a medical image to be displayed on the display device 26, which is the medical image acquired in the image acquisition step S10. After the image processing step S12, the process proceeds to a display signal generation step S26. In the image analysis step S14, the medical image acquired in the image acquisition step S10 is analyzed. After the image analysis step S14, the process proceeds to a treatment progress information generation step S24.

In an interpretation information acquisition step S16, the interpretation information acquisition unit 40 acquires interpretation information such as an interpretation report of the examination target subject. After the interpretation information acquisition step S16, the process proceeds to an interpretation information analysis step S18. In the interpretation information analysis step S18, the interpretation information analysis unit 42 analyzes the interpretation information acquired in the interpretation information acquisition step S16 to acquire the first treatment information. After the interpretation information analysis step S18, the process proceeds to the treatment progress information generation step S24. The interpretation information acquisition step S16 and the interpretation information analysis step S18 are examples of components of a treatment information acquisition step.

In a medical record information acquisition step S20, the medical record information acquisition unit 44 acquires an electronic medical record of the examination target subject. After the medical record information acquisition step S20, the process proceeds to a medical record information analysis step S22. In the medical record information analysis step S22, the medical record information analysis unit 46 analyzes the electronic medical record of the examination target subject acquired in the medical record information acquisition step S20 to acquire the second treatment information. After the medical record information analysis step S22, the process proceeds to a treatment progress information generation step S24. The medical record information acquisition step S20 and the medical record information analysis step S22 are examples of components of the treatment information acquisition step.

In the treatment progress information generation step S24, the treatment progress information generation unit 48 generates treatment progress information using the processing result of the image analysis step S14, the first treatment information acquired in the interpretation information analysis step S18, and the second treatment information acquired in the medical record information analysis step S22. After the treatment progress information generation step S24, the process proceeds to the display signal generation step S26.

In the display signal generation step S26, the display signal output unit 34 generates a display signal representing the current image using the processing result of the image processing step S12. In addition, in the display signal generation step S26, the display signal output unit 34 generates a display signal representing the treatment progress information using the processing result of the treatment progress information generation step S24. After the display signal generation step S26, the process proceeds to a display signal output step S28.

In the display signal output step S28, the display signal output unit 34 causes the display device 26 to output the display signal representing the current image and the display signal representing the treatment progress information, which are generated in the display signal generation step S26. The display device 26 displays a current image and treatment progress information.

### Specific Example of Treatment Progress Information

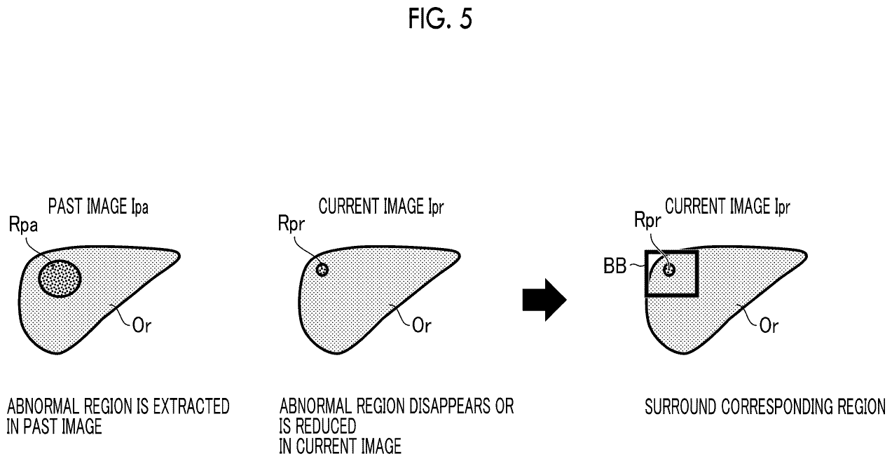

FIG. 5 is a schematic diagram of treatment progress information according to a first specific example. A past image Ipa shown in FIG. 5 is an image generated in an examination performed in the past for an examination target subject of a current image Ipr. An organ Or shown in the past image Ipa is the same as an organ Or shown in the current image Ipr. The organ Or is understood as an example of a first anatomical structure.

In the past image Ipa, an abnormal region Rpa in the organ Or is extracted. On the other hand, as a result of the successful progress of the treatment, the abnormal region Rpr in the organ Or has been reduced in the current image Ipr. In this case, in the current image Ipr, it may be difficult to recognize the abnormal region Rpr.

FIG. 5 shows an example in which the abnormal region Rpr of the organ Or is reduced in the current image Ipr, but the same applies to a case where the abnormal region Rpr has disappeared, such as a case where the abnormal region Rpr has been excised.

Therefore, a treatment region in the current image Ipr, which is an image to be interpreted, is displayed in an easy-to-understand manner using the information obtained from the current image Ipr, the information obtained from the past interpretation report, and the information obtained from the electronic medical record. Specifically, treatment progress information is displayed on the current image Ipr.

A bounding box BB shown in FIG. 5 is an example of treatment progress information, and has a position and a shape surrounding the abnormal region Rpa in the past image Ipa. That is, the bounding box BB is disposed at a position of the current image Ipr corresponding to a position of the abnormal region Rpa in the past image Ipa and has a size corresponding to a size of the abnormal region Rpa in the past image Ipa.

Specifically, the bounding box BB has a square that has a centroid position in the current image Ipr that matches a centroid position of the abnormal region Rpa in the past image Ipa and a square whose size is inscribed with the abnormal region Rpa in the past image Ipa is applied to the bounding box BB.

As the bounding box BB, various shapes such as a quadrangle other than a square such as a triangle or a rectangle, a polygon such as a pentagon, a circle, an ellipse, or any figure may be applied. Further, the type, width, and color of the line constituting the bounding box can be optionally defined.

The current image Ipr shown in FIG. 5 is an example of a first diagnosis target image included in the first medical image.

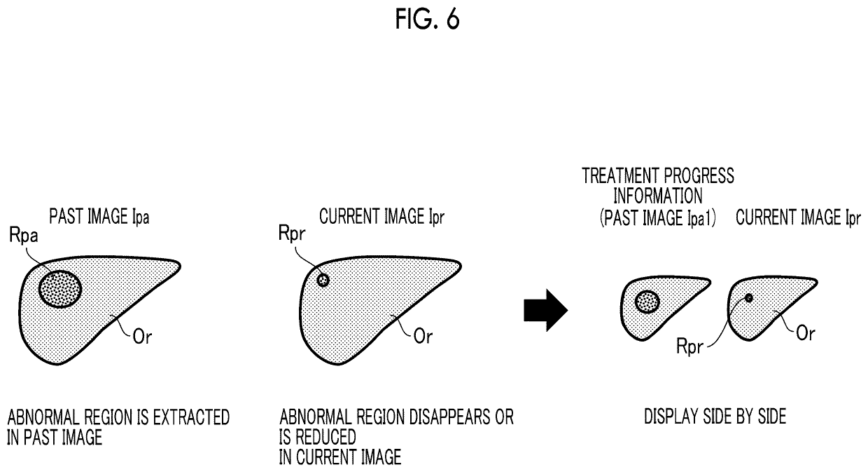

FIG. 6 is a schematic diagram of treatment progress information according to a second specific example. A past image Ipa1 shown in FIG. 6 is an example of treatment progress information and is displayed side by side with the current image Ipr. FIG. 6 shows an example in which the current image Ipr is reduced and the past image Ipa1 is reduced to the same size as the reduced current image Ipr is displayed on the screen on which only the current image Ipr is displayed. Note that the past image Ipa1, which is the treatment progress information, may have a size larger than the current image Ipr or may have a size smaller than the current image Ipr. In addition, the past image Ipa1, which is the treatment progress information, may be displayed in a separate window from the current image Ipr.

As the treatment progress information, a first trimmed image obtained by trimming the same region as a region of a lesion at a position of the lesion for which an instruction of a treatment has been provided in the past in the current image Ipr may be applied. The first trimmed image, which is the treatment progress information, may be disposed at the position of the past image Ipa1 shown in FIG. 6.

In a case where a feature of the treatment location cannot be recognized in the first trimmed image, a second trimmed image of a region corresponding to the treatment location of the first trimmed image is generated in the past image Ipa before the treatment is performed, and the second trimmed image may be used as the treatment progress information.

In a case where the past image Ipa1 is applied to the treatment progress information, a slice position showing an organ for which an instruction of a treatment has been provided in the past in a slice image group including a plurality of slice images may be applied to the treatment progress information. The slice position showing the organ for which an instruction of a treatment has been provided in the past, which is described in the embodiment, is an example of information for designating the position of the reconstructed image in a reconstructed image group including a plurality of reconstructed images.

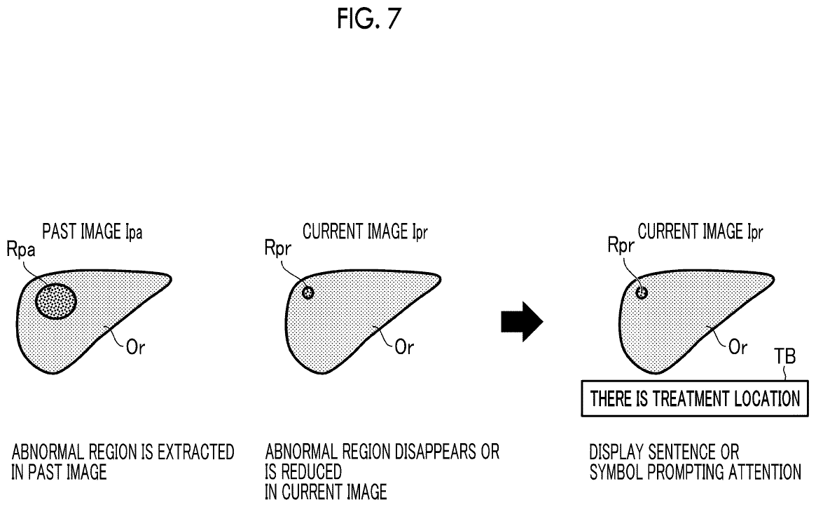

FIG. 7 is a schematic diagram of treatment progress information according to a third specific example. A text box TB shown in FIG. 7 is an example of treatment progress information, and includes character information such as a sentence prompting attention that the abnormal region Rpa was present in the past image Ipa. A symbol or the like may be used in the text box TB. The character information is understood as text data.

In the text box TB shown in FIG. 7, a line type, a width, and a color applied to a frame surrounding the character information can be optionally defined. In the text box TB shown in FIG. 7, an aspect in which a frame surrounding the character information is not included may be applied.

In a case where there are a plurality of pieces of treatment progress information, a display mode in which each piece of treatment progress information is distinguished is preferable. For example, in a case where there are two or more bounding boxes BB shown in FIG. 5, at least one of a type, a width, or a color of a line constituting each of the two or more bounding boxes BB may be different from each other.

### Specific Example of Acquisition of Treatment Information

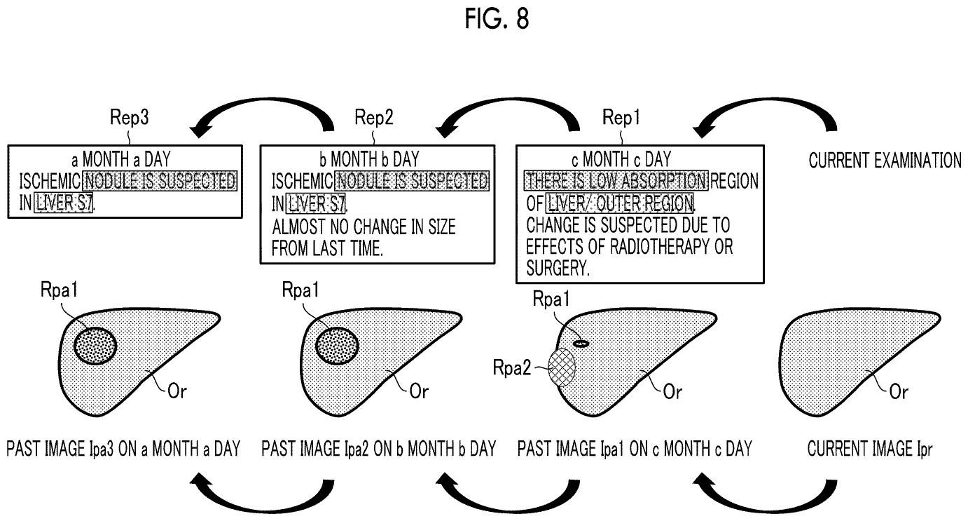

FIG. 8 is a schematic diagram of acquisition of treatment information according to the first specific example. FIG. 8 schematically shows processing of acquiring treatment information from a past interpretation report and a past image Ipa in a case of creating an interpretation report of the current image Ipr in the current examination. Note that a month a day, b month b day, and c month c day represent any dates; the oldest date is a month a day, and the newest date is c month c day. b month b day is a date between a month a day and c month c day.

In a case of tracing back the past interpretation report, a name of an organ for which an instruction of a treatment has been provided in the past, a name of a lesion for which the instruction of the treatment has been provided in the past, a position of the lesion for which the instruction of the treatment has been provided in the past, a treatment method performed in the past, and the like are ascertained. Examples of a position of a lesion include a region into which an organ is divided. Examples of a treatment method include operative method names of surgery, chemotherapy, and radiotherapy.

For example, in an interpretation report Rep1 on c month c day, which was created in the previous examination, a liver is present as the organ name, an outer region is present as the position of the lesion, and a low absorption region is present as the lesion name. Further, the interpretation report Rep1 includes radiotherapy and surgery as treatment methods.

Further, in an interpretation report Rep2 on b month b day, which was created in the examination before the previous examination, a liver is present as the organ name, S7 is present as the position of the lesion, and an ischemic nodule is present as the lesion name. In an interpretation report Rep3 on a month a day, which was created in the examination before the previous two examinations, a liver is present as the organ name, S7 is present as the position of the lesion, and an ischemic nodule is present as the lesion name.

In a case where the past image Ipa is traced back based on the past interpretation report Rep, the position, the shape, and the property of each of the abnormal region Rpa1 and the abnormal region Rpa2 in the past image Ipa, which are abnormal regions not present in the current image Ipr, are ascertained. The interpretation report Rep is a general term for the interpretation report Rep1 and the like. In addition, the past image Ipa is a general term for the past image Ipa1 and the like.

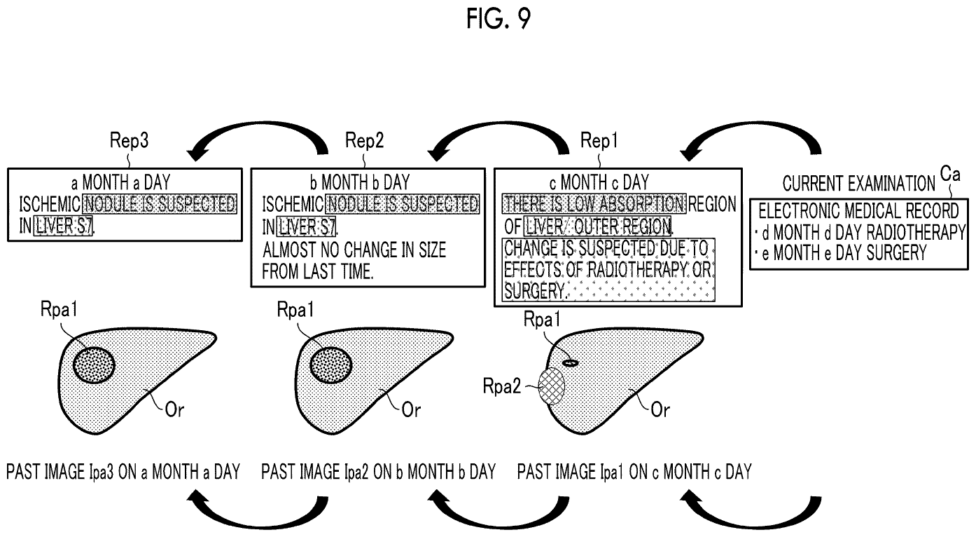

FIG. 9 is a schematic diagram of acquisition of treatment information according to the second specific example. FIG. 9 shows an aspect in which treatment information is acquired using an electronic medical record Ca stored in the hospital information system 16 in addition to information stored in the radiology information system 14.

The electronic medical record Ca contains all the information regarding the treatment in the examination target subject, and in a case where the examination target subject has a treatment history, items related to the treatment can be extracted from the electronic medical record Ca. In the electronic medical record Ca shown in FIG. 9, information regarding the radiotherapy on d month d day and information regarding the surgery on e month e day are described. In addition, d month d day and e month e day represent any dates.

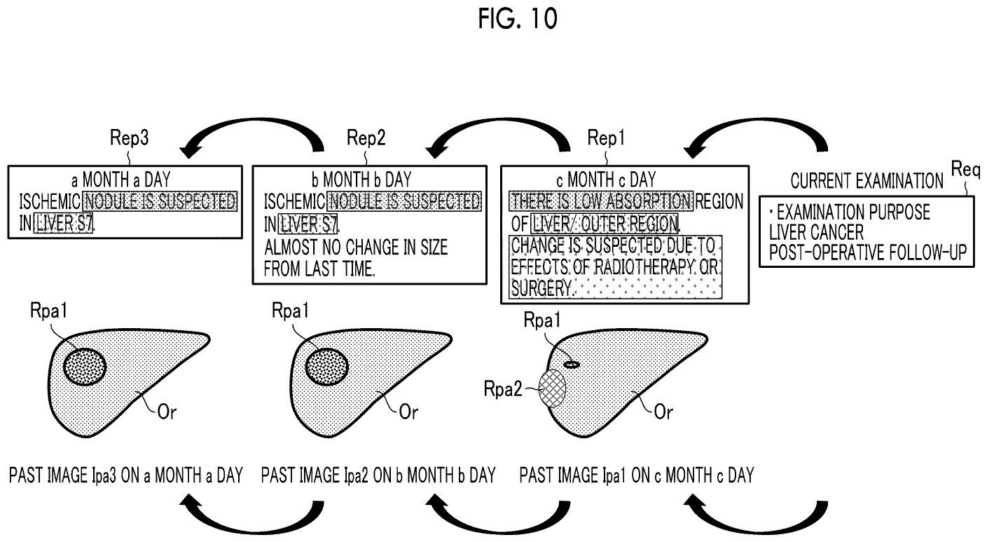

FIG. 10 is a schematic diagram of acquisition of treatment information according to the third specific example. FIG. 10 shows an example in which treatment information is acquired from an examination request Req for the current examination stored in the radiology information system 14. FIG. 10 shows an example in which an examination purpose of performing liver cancer post-operative follow-up is described in the examination request Req for the current examination. That is, the treatment information can be acquired based on not only past information such as a past interpretation report but also current information such as the examination purpose included in the examination request Req for the current examination. The examination request Req described in the embodiment is an example of request information.

### Effects of Image Diagnosis Assisting Method According to First Embodiment

An image diagnosis assisting method according to a first embodiment can obtain the following effects.

[1]

For the current image Ipr, treatment progress information such as the bounding box BB shown in FIG. 5 is generated based on treatment information regarding the examination target subject of the current image Ipr. The treatment progress information is displayed on the screen on which the current image Ipr is displayed. Accordingly, even in a case where the past treatment location has been reduced or disappeared in the current image Ipr, it is possible to ascertain the past treatment location in the current image Ipr.

[2]

As treatment information, past information, such as an analysis result of a past image, first treatment information obtained by analyzing a past interpretation report, and second treatment information obtained by analyzing an electronic medical record, is applied. Accordingly, treatment progress information based on the past information can be generated.

[3]

As treatment information, a name of an organ for which an instruction of a treatment has been provided in the past, a name of a lesion for which the instruction of the treatment has been provided in the past, a position of the lesion for which the instruction of the treatment has been provided in the past, a treatment method performed in the past, and the like are applied. Accordingly, treatment progress information based on the name or the like of the organ for which the instruction of the treatment has been provided in the past is generated.

[4]

As treatment information, the examination purpose or the like included in the current examination request Req is applied. Accordingly, treatment progress information based on current information can be generated.

[5]

As treatment progress information, a bounding box BB representing the past abnormal region Rpa and a text box TB including sentences prompting attention that the abnormal region Rpa was present in the past image Ipa1 and the past image Ipa are applied. Accordingly, it is possible to ascertain the position, the shape, the property, or the like of the past lesion that is reduced or disappeared in the current image Ipr.

[6]

As treatment progress information, a first trimmed image obtained by trimming the same region as the past abnormal region Rpa in the current image Ipr is applied. Accordingly, it is possible to ascertain the position, the shape, the property, or the like of the same region as the past abnormal region Rpa in the current image Ipr.

### Modification Example of Image Diagnosis Assisting Method

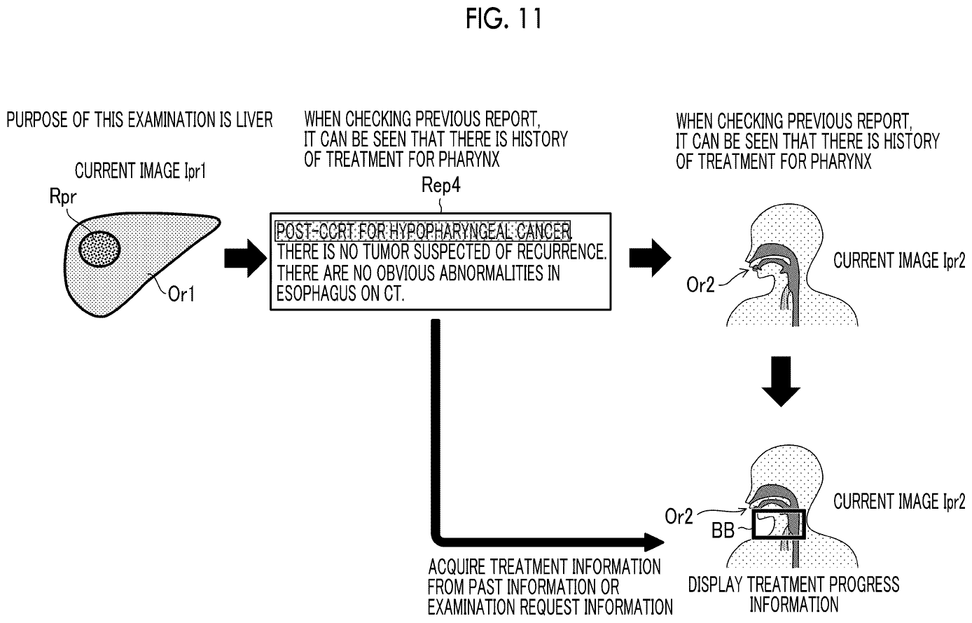

FIG. 11 is a schematic diagram of an image diagnosis assisting method according to a modification example. FIG. 11 schematically shows a case where an anatomical structure of a current examination target and the anatomical structure included in the treatment information are different from each other. FIG. 11 shows a case where a first anatomical structure Or1 of the current examination target is a liver and a second anatomical structure Or2 included in the treatment information is a pharynx.

In a case where the abnormal region Rpr is found in a current image Ipr1 that shows the liver included in the current image Ipr generated in the current examination, checking a past interpretation report Rep4 reveals a treatment history for the pharynx, including post-CCRT for hypopharyngeal cancer.

In a case where a current image Ipr2 that shows the pharynx is present in a current image group including a plurality of current images including the current image Ipr1 that shows the liver, the display is moved from the current image Ipr1 to the current image Ipr2 and the past image Ipa and the current image Ipr2 are compared.

That is, the interpretation information acquisition unit 40 shown in FIG. 2 acquires the past interpretation report Rep4 as treatment information. The interpretation information analysis unit 42 analyzes the past interpretation report Rep4. The treatment progress information generation unit 48 generates the bounding box BB surrounding the pharynx which is the second anatomical structure Or2 as treatment progress information based on treatment information of a hypopharyngeal cancer, which is an analysis result of the past interpretation report Rep4. For the current image Ipr2 including the pharynx, the bounding box BB is generated as treatment progress information.

On the other hand, in a case where the current image Ipr2 that shows the pharynx is not present in the current image group including the current image Ipr1 that shows the liver, acquisition of treatment information, analysis of treatment information, and generation of treatment progress information are not performed, and the treatment progress information for the current image Ipr2 that shows the pharynx is hidden. Note that the current image Ipr2 that shows the pharynx described in the embodiment is a second diagnosis target image including a second anatomical structure, and is an example of a second diagnosis target image included in the first medical image.

### Effects of Image Diagnosis Assisting Method According to Second Embodiment

An image diagnosis assisting method according to a second embodiment can obtain the following effects.

In a case where the first anatomical structure Or1 of the current examination target is different from the second anatomical structure Or2 in which the treatment history is present in the past, in the current image group including the current image Ipr1, in a case where there is the current image Ipr2 that shows the second anatomical structure Or2 for which a treatment history is present in the past, treatment progress information for the current image Ipr2 is generated. The treatment progress information for the current image Ipr2 is displayed on the screen on which the current image Ipr2 is displayed. Accordingly, it is possible to ascertain the position, the shape, the property, or the like of the past abnormal region Rpa for the second anatomical structure Or2 different from the first anatomical structure Or1 that is the current examination target.

### Variation of Medical Image

The slice image applied to the current image Ipr and the past image Ipa may be an axial cross section image, a coronal cross section image, or a sagittal cross section image. The slice thickness of the slice image can be optionally defined.

The current image Ipr and the past image Ipa may be a two-dimensional reconstructed image or a three-dimensional reconstructed image. The reconstruction conditions are not particularly limited.

The technical scope of the present invention is not limited to the scope described in the above embodiment. The configurations and the like in each embodiment can be appropriately combined between the respective embodiments without departing from the spirit of the present invention.

### EXPLANATION OF REFERENCES

10: hospital system
12: examination apparatus
14: radiology information system
16: hospital information system
16A: automatic reception system
16B: electronic medical record system
16C: hospitalization/discharge management system
16D: medical accounting system
16E: pharmacy management system
16F: medical care reservation system
18: image storage communication system
20: report creation system
21: report creation terminal device
22: server device
23: database
24: display terminal
26: display device
27: input device
30: image acquisition unit
32: image processing unit
34: display signal output unit
36: image analysis unit
40: interpretation information acquisition unit
42: interpretation information analysis unit
44: medical record information acquisition unit
46: medical record information analysis unit
48: treatment progress information generation unit
52: processor
54: computer-readable medium
56: communication interface
58: input/output interface
60: bus
62: memory
64: storage
70: image acquisition program
72: image processing program
74: image analysis program
76: display signal generation program
80: interpretation information acquisition program
82: interpretation information analysis program
84: medical record information acquisition program
86: medical record information analysis program
88: treatment progress information generation program
BB: bounding box
Ipa: past image
Ipa1: past image
Ipa2: past image
Ipa3: past image
Ipr: current image
Ipr1: current image
Ipr2: current image
Or: organ
Or1: first anatomical structure
Or2: second anatomical structure
Rep1: interpretation report
Rep2: interpretation report
Rep3: interpretation report
Rep4: interpretation report
Rpa: abnormal region in past image
Rpa1: abnormal region in past image
Rpa2: abnormal region in past image
Rpr: abnormal region in current image
TB: text box
S10 to S28: each step of image diagnosis assisting method

What is claimed is:

1. An image diagnosis assisting device comprising:
one or more processors; and
one or more memories in which instructions to be executed by the one or more processors are stored,
wherein the one or more processors are configured to:
acquire a first medical image generated by imaging a subject in a latest examination;
acquire treatment information regarding a treatment of the subject;
generate treatment progress information regarding a past treatment location in the subject based on the treatment information; and
perform control to display the treatment progress information on a screen on which the first medical image is displayed.

2. The image diagnosis assisting device according to claim 1,
wherein the one or more processors are configured to acquire information regarding the past treatment location of the subject as the treatment information.

3. The image diagnosis assisting device according to claim 2,
wherein the one or more processors are configured to acquire, as the information regarding the past treatment location of the subject, character information including a description regarding the past treatment location of the subject.

4. The image diagnosis assisting device according to claim 1,
wherein the one or more processors are configured to acquire request information for the latest examination of the subject as the treatment information.

5. The image diagnosis assisting device according to claim 4,
wherein the one or more processors are configured to acquire, as the request information for the latest examination of the subject, character information including a description regarding a purpose of the latest examination for the subject.

6. The image diagnosis assisting device according to claim 1,
wherein the one or more processors are configured to acquire, as the treatment information, at least one of a name of an anatomical structure for which an instruction of a treatment has been provided in the past, a name of a lesion for which the instruction of the treatment has been provided in the past, a position of the lesion for which the instruction of the treatment has been provided in the past, or a treatment method that has been performed in the past.

7. The image diagnosis assisting device according to claim 1,
wherein the one or more processors are configured to display the treatment progress information on a first diagnosis target image included in the first medical image, the first diagnosis target image including a first anatomical structure to be diagnosed in the latest examination.

8. The image diagnosis assisting device according to claim 7,
wherein the one or more processors are configured to generate, as the treatment progress information, information for designating, in a reconstructed image group including a plurality of two-dimensional reconstructed images, a position of the reconstructed image including the past treatment location.

9. The image diagnosis assisting device according to claim 7,
wherein the one or more processors are configured to apply segmentation processing of the first anatomical structure or labeling processing of the first anatomical structure to the first medical image to specify the past treatment location in the first diagnosis target image.

10. The image diagnosis assisting device according to claim 7,
wherein the one or more processors are configured to:
acquire a second medical image generated by imaging a subject in a past examination; and
apply segmentation processing of the first anatomical structure or labeling processing of the first anatomical structure to the second medical image to specify the past treatment location in the second medical image.

11. The image diagnosis assisting device according to claim 1,
wherein the one or more processors are configured to generate, as the treatment progress information, a first trimmed image in which a region corresponding to the past treatment location in a first diagnosis target image included in the first medical image is trimmed, the first diagnosis target image including a first anatomical structure to be diagnosed in the latest examination.

12. The image diagnosis assisting device according to claim 11,
wherein the one or more processors are configured to:
analyze the first trimmed image;
in a case where a feature in an image related to the treatment location is not recognizable in the first trimmed image, generate a second trimmed image in which the region corresponding to the treatment location is trimmed from a past medical image of the subject; and
display the second trimmed image in the region corresponding to the past treatment location in the first diagnosis target image.

13. The image diagnosis assisting device according to claim 1,
wherein the one or more processors are configured to:
in a case where a first anatomical structure to be examined in the latest examination is different from a second anatomical structure which is the past treatment location of the subject,
acquire the treatment information for the second anatomical structure;
generate the treatment progress information for the second anatomical structure based on the treatment information for the second anatomical structure; and
display the treatment progress information for the second anatomical structure on a second diagnosis target image included in the first medical image, the second diagnosis target image including the second anatomical structure.

14. The image diagnosis assisting device according to claim 1,
wherein the one or more processors are configured to:
generate a plurality of pieces of the treatment progress information; and
apply a different display mode to each of the plurality of pieces of treatment progress information.

15. An operation method of an image diagnosis assisting device to which a computer is applied, the operation method comprising:
by the image diagnosis assisting device,
acquiring a first medical image generated by imaging a subject in a latest examination;
acquiring treatment information regarding a treatment of the subject;
generating treatment progress information regarding a past treatment location in the subject based on the treatment information; and
performing control to display the treatment progress information on a screen on which the first medical image is displayed.

16. A non-transitory, computer-readable tangible recording medium which records thereon a program for causing, when read by a computer, the computer to implement:
a function of acquiring a first medical image generated by imaging a subject in a latest examination;
a function of acquiring treatment information regarding a treatment of the subject;
a function of generating treatment progress information regarding a past treatment location in the subject based on the treatment information; and
a function of performing control to display the treatment progress information on a screen on which the first medical image is displayed.
